(19) United States
(12) Patent Application Publication
(10) Pub. No.: US 2026/0094676 A1
(43) Pub. Date: Apr. 2, 2026

(54) METHOD AND APPARATUS FOR PREDICTING DRUG BIOAVAILABILITY

(71) Applicant: LIVERPOOL JOHN MOORES UNIVERSITY, Liverpool (GB)

(72) Inventors: Amos Akintayo FATOKUN, Liverpool (GB); Sunday Olakunle IDOWU, Ibadan (NG); Morenikeji Ayodele ADEYEMO, Ibadan (NG)

(21) Appl. No.: 19/110,620

(22) PCT Filed: Sep. 12, 2023
(86) PCT No.: PCT/GB2023/052364

§ 371 Date: Mar. 11, 2025

(30) Foreign Application Priority Data
Sep. 12, 2022 (GB) 2213304.5

(51) Int. Cl. G16C 20/30

(52) U.S. Cl. CPC G16C 20/30

(57) ABSTRACT

The present invention relates to a method and apparatus for predicting the effect of the co-administration of a secondary active compound on the bioavailability of a primary active compound.

### TECHNICAL FIELD OF THE INVENTION

The present invention relates to a method and apparatus for predicting the effect of the co-administration of a secondary active compound on the bioavailability of a primary active compound.

### BACKGROUND TO THE INVENTION

The drug discovery and development process is extremely complex and requires the collaborative effort of an interdisciplinary team of scientists and enormous financial investment. It involves the identification and validation of a disease target through scientific approaches such as genomic and proteomic technology, and screening of various chemical or biological compounds through different high throughput assay techniques with the purpose of finding lead drug candidates for further development (Rick, 2009). The success of any drug discovery project in translating a new chemical entity into promising drug candidate is however dependent on reliable and accurate prediction of pharmacokinetic (PK) parameters i.e. absorption, distribution, metabolism and toxicity (ADMET) at the early stage of drug discovery investigations. As such, many investigative compounds with potential for therapeutic action have however failed to make it to the market after many years of research; while a few that made it to the market had to be withdrawn due to poor biopharmaceutical properties and problem of intolerable toxicity as a result of inaccurate prediction of their pharmacokinetics prior to drug development. This has led to significant high attrition rate for the drug discovery process accompanied by huge financial loss. In order to reduce this failure rate, suggestions have been made to change from the traditional approach to newer approaches that allow early prediction of the pharmacokinetic and biopharmaceutical properties of these drug molecules even before drug development stage. Several physicochemical parameters such as solubility, ionization constant, polar surface area, lipophilicity and permeability etc. have been found to influence drug biopharmaceutical properties (Lipinski, 2000). Hence, these properties must be reliably and accurately predicted during the early stage of drug discovery project in order to reduce attrition rate (Kern et al., 2003; Penzotti et al., 2004). Of these physicochemical parameters, lipophilicity has been proven to have the major predictive value on ADMET properties and pharmacological activity since molecules must be transported across the bio-membrane in order to interact with receptors towards eliciting biological action (Serda et al., 2012). As a result of its close correlation with permeability and drug solubility (Cross et al., 2003); it has been widely put to use in several pharmacokinetic models (Kaliszan et al., 2003). A number of models have been adopted in estimating this parameter towards prediction of the drug PK but with varying in-vitro-in-vivo correlation.

Unfortunately, there still remains an unmet need to fully account for the amphiphilic chemistry of the biological membrane.

It is an object of the present invention to address one or more of the issues associated with the prior art methods of assessing drug-drug interactions. It is also an object of the present invention to provide a method which can assess drug-drug interactions whilst taking account of the amphiphilic chemistry of the biological membrane.

### SUMMARY OF THE INVENTION

In accordance with an aspect of the present invention, A method for predicting the effect of the co-administration of a secondary active compound on the bioavailability of a primary active compound in an individual, the method comprising:
(a) calculating the solubility interaction quotient (Qₛᵢ) for the combination of primary and secondary active compounds according to the formula:

$${Q}_{si} = \frac{{ICHI}_{1}}{{ICHI}_{2}}$$

wherein ICHI₁ is the isocratic chromatographic hydrophobicity index (ICHI) of the primary active compound and ICHI₂ is the ICHI of the secondary active compound; and
(b) characterising the predicted effect of the co-administration of the secondary active compound on the bioavailability of the primary active compound based on the calculated Qₛᵢ value as either:
   (i) reducing the bioavailability;
   (ii) enhancing the bioavailability; or
   (iii) having substantially no effect on the bioavailability.

The ICHI values of the primary and secondary active compounds are preferably measured using a Leucaena Oil Device (LOD). The LOD was designed for lipophilicity measurements and was created and reported to provide a more accurate simulation of the bio-membrane (Idowu et al., 2009). This tool advantageously offers potential advantage as a solution option to the problem of high attrition in the drug discovery process.

Other oils, preferably other seed oils, with similar properties to Leucaena oil can be used instead of Leucaena oil in devices for measuring ICHI values for use in the invention. For example, oils with similar levels of amphiphilic lecithin to Leucaena oil in their crude and refined forms are expected to have similar biomimetic attributes to Leucacna oil. In particular, refined oils with 0.25%-0.75% w/w of lecithin are expected to be suitable for use in the invention. Oils with a hydrophobicity equivalent to about that produced by 5% v/v of liquid paraffin as plate-coating solution are expected to be suitable for use in the invention.

The predicted effect of the co-administration of the secondary active compound on the bioavailability of the primary active compound may be characterised as:
(i) reducing the bioavailability if Qₛᵢ is greater than about 2;
(ii) enhancing the bioavailability if Qₛᵢ in the range of about 1 to about 2; or
(iii) having substantially no effect on the bioavailability if Qₛᵢ is equal to about 1.

Alternatively, the predicted effect of the co-administration of the secondary active compound on the bioavailability of the primary active compound may be characterised as:
(i) reducing the bioavailability if Qₛᵢ is greater than about 2.3;
(ii) enhancing the bioavailability if Qₛᵢ in the range of about 1.4 to about 2.2; or
(iii) having substantially no effect on the bioavailability if Qₛᵢ less than about 1.3.

Most preferably, the predicted effect of the co-administration of the secondary active compound on the bioavailability of the primary active compound may be characterised as:
(i) reducing the bioavailability if Qₛᵢ≥about 2.27;
(ii) enhancing the bioavailability if about 1.35≤Qₛᵢ<about 2.27; or
(iii) having substantially no effect on the bioavailability if Qₛᵢ<about 1.35.

The method is particularly suited when the primary and/or secondary active compounds comprise small molecules. It will be apparent to the skilled addressee that a small molecule (in medicinal terms) typically has a molecular weight of ≤900 Da.

The primary active compound may be lipophilic.

The method of analysis will preferably be computer-implemented and will comprise input means, a central processing unit (CPU) and some form of visual display for presenting the result to a user.

In accordance with another aspect of the present invention, there is provided a Leucaena oil device for use in predicting the effect of the co-administration to a subject of a secondary active compound on the bioavailability of a primary active compound in an individual.

Preferably, the Leucaena oil device is used to measure ICHI values for the primary and secondary active compounds.

The primary and/or secondary active compounds may comprise small molecules which may additionally be lipophilic.

In accordance with a further aspect of the present invention, there is provided an apparatus for predicting the effect of the co-administration of a secondary active compound on the bioavailability of a primary active compound in an individual, the apparatus comprising:
(a) a Leucaena oil device;
(b) means for calculating the Qₛᵢ for the combination of primary and secondary active compounds according to the formula:

$${Q}_{si} = \frac{{ICHI}_{1}}{{ICHI}_{2}}$$

wherein ICHI₁ is the isocratic chromatographic hydrophobicity index (ICHI) of the primary active compound and ICHI₂ is the ICHI of the secondary active compound as measured by the Leucaena oil device; and
(c) means for characterising the predicted effect of the co-administration of the secondary active compound on the bioavailability of the primary active compound based on the calculated Qₛᵢ value as either:
   (i) reducing the bioavailability;
   (ii) enhancing the bioavailability; or
   (iii) having substantially no effect on the bioavailability.

The predicted effect of the co-administration of the secondary active compound on the bioavailability of the primary active compound may be characterised as:
(i) reducing the bioavailability if Qₛᵢ is greater than about 2;
(ii) enhancing the bioavailability if Qₛᵢ in the range of about 1 to about 2; or
(iii) having substantially no effect on the bioavailability if Qₛᵢ is equal to about 1.

Alternatively, the predicted effect of the co-administration of the secondary active compound on the bioavailability of the primary active compound is characterised as:
(i) reducing the bioavailability if Qₛᵢ is greater than about 2.3;
(ii) enhancing the bioavailability if Qₛᵢ in the range of about 1.4 to about 2.2; or
(iii) having substantially no effect on the bioavailability if Qₛᵢ less than about 1.3.

Preferably, the predicted effect of the co-administration of the secondary active compound on the bioavailability of the primary active compound is characterised as:
(i) reducing the bioavailability if Qₛᵢ≥about 2.27;
(ii) enhancing the bioavailability if about 1.35≤Qₛᵢ<about 2.27; or
(iii) having substantially no effect on the bioavailability if Qₛᵢ<about 1.35.

The primary and/or secondary active compounds analyzed by the apparatus may comprise small molecules and may be lipophilic.

It will be apparent to the skilled addressee that the apparatus may further comprise a computer or central processing unit utilized to calculate the Qₛᵢ value. Other features, such as visual display unit may also be provided with the apparatus.

### DETAILED DESCRIPTION OF THE INVENTION

Embodiments of the invention are described below, by way of example only, with reference to the accompanying figures in which:

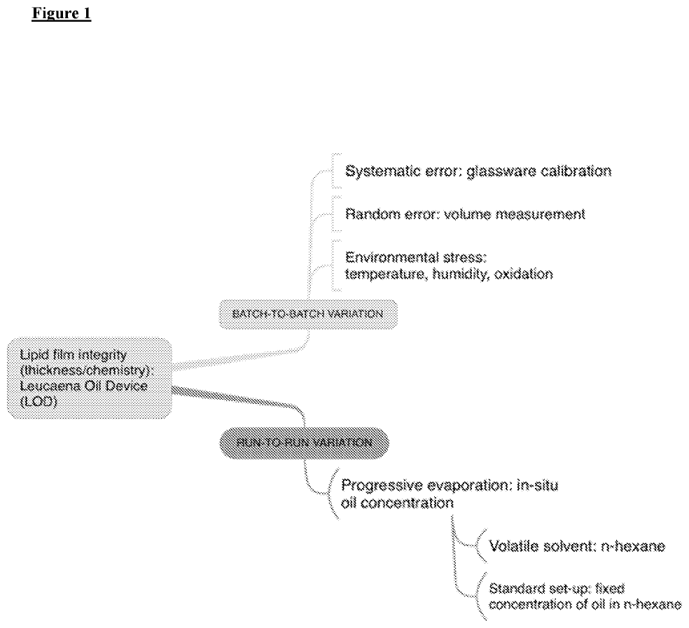

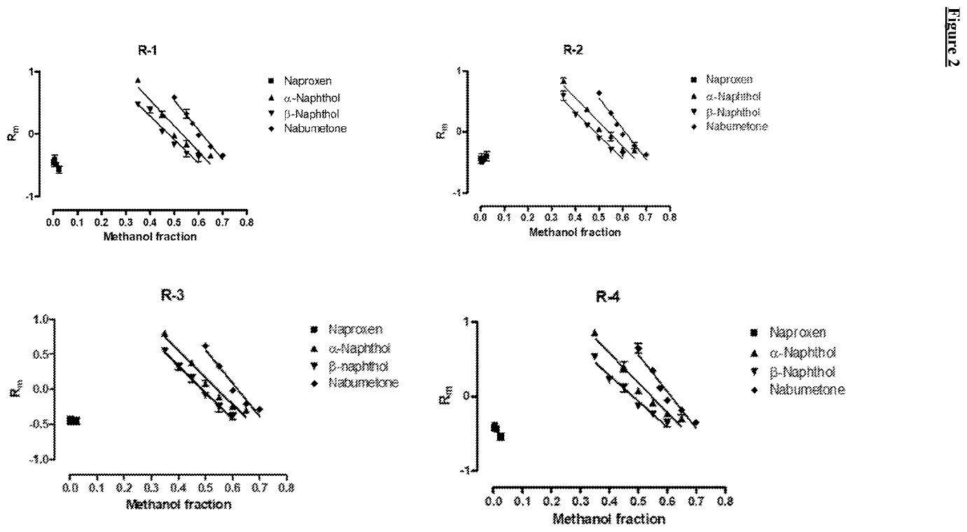

FIG. 1 is an Ishikawa (Fishbone) diagram showing the inter-relationships between the variable inputs in the lipid device creation process and its stability on storage;

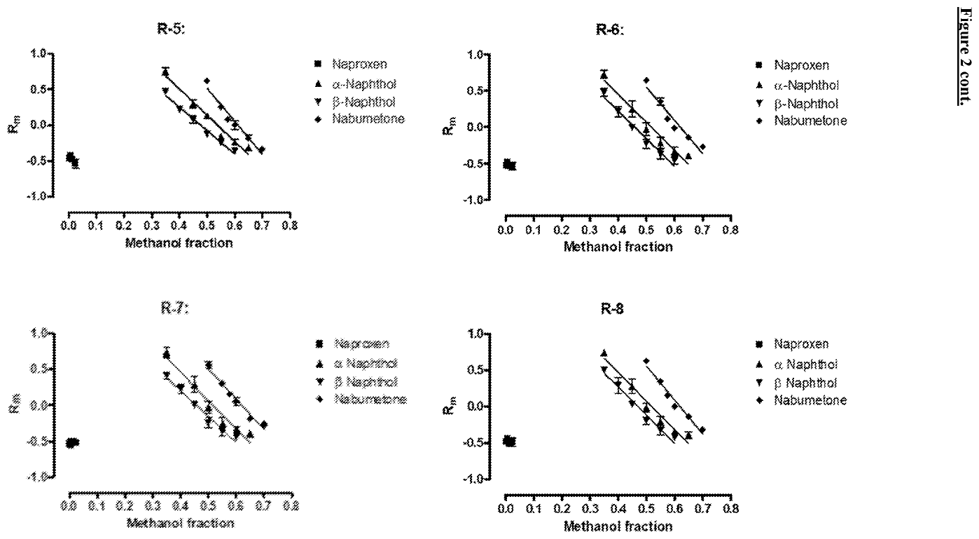

FIG. 2 arc plot graphs of Rm versus methanol fraction for naproxen, α-naphthol, β-naphthol and nabumetone from 1-10 runs;

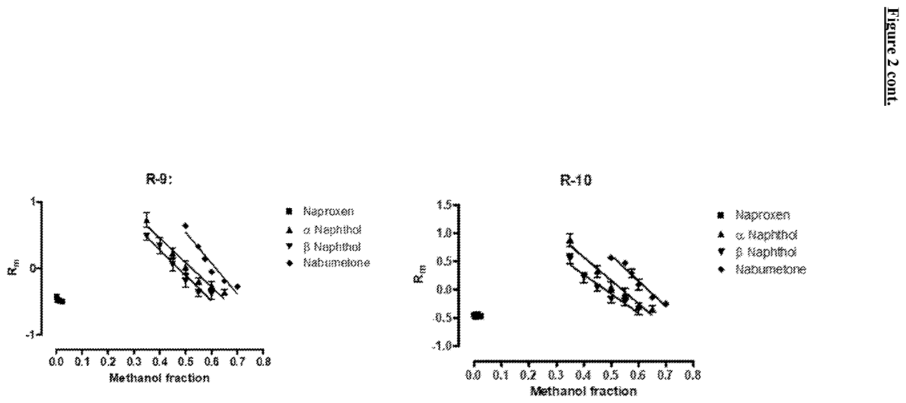

FIG. 3 are plot graphs of Rm_{w} and specific hydrophobic surface area for naproxen, α-naphthol, β-naphthol and nabumetone from 1-10 runs;

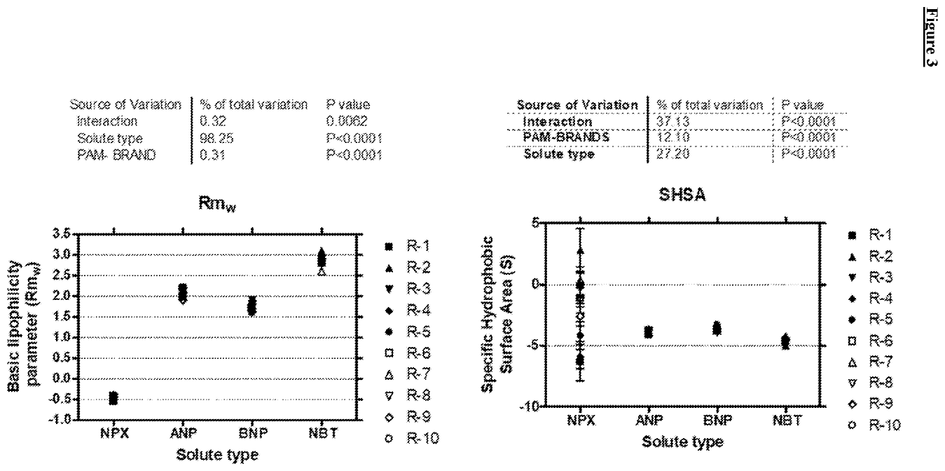

FIG. 4 is a bar graph of ICHI for naproxen, α-naphthol, β-naphthol and nabumetone from 1-10 runs;

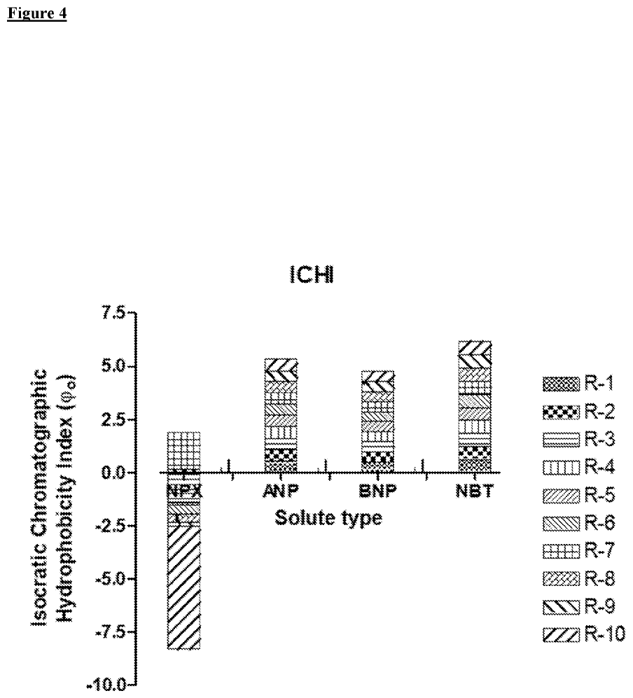

FIG. 5 are plot graphs of Rm versus organic modifier fraction for α-naphthol, β-naphthol and nabumetone;

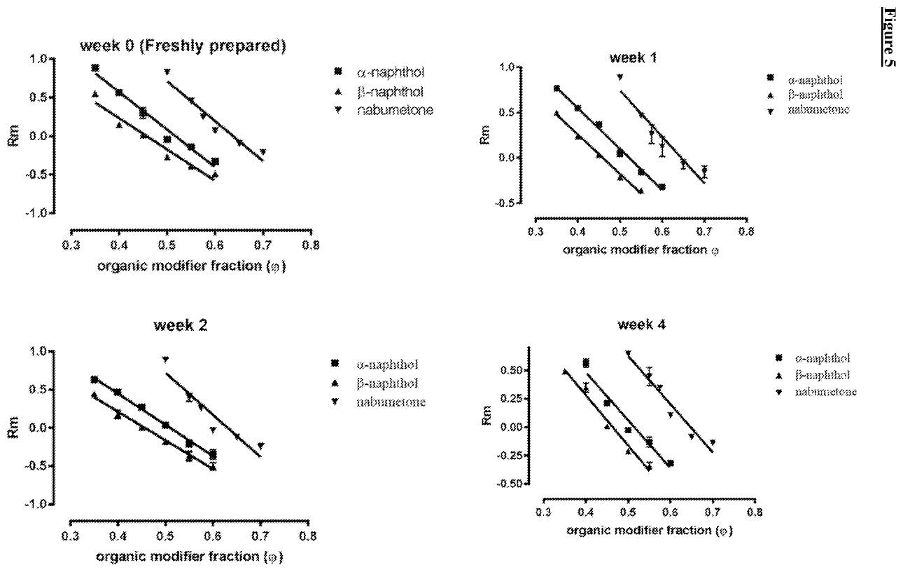

FIG. 6 are plot graphs of Rm_{w} and specific hydrophobic surface area for α-naphthol, β-naphthol and nabumetone from 0-52 weeks;

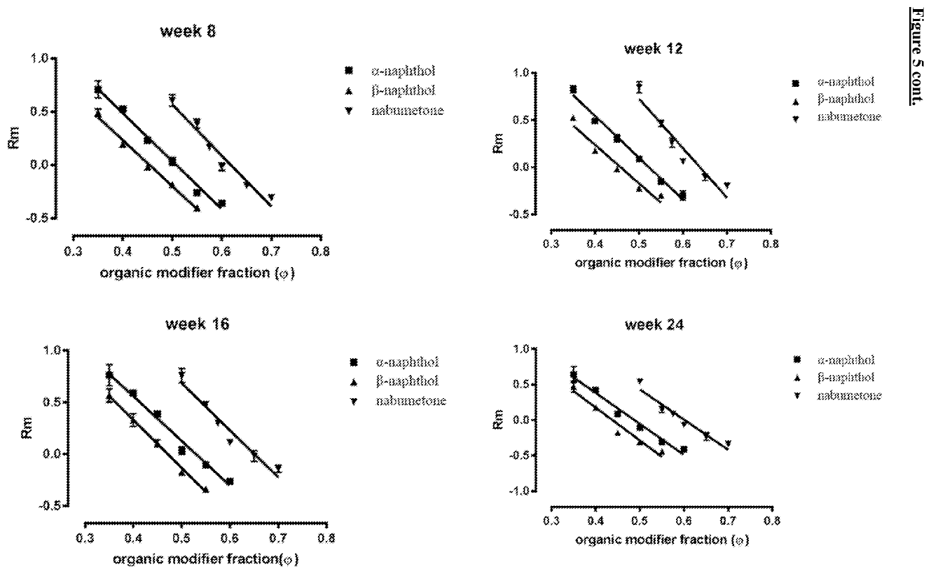

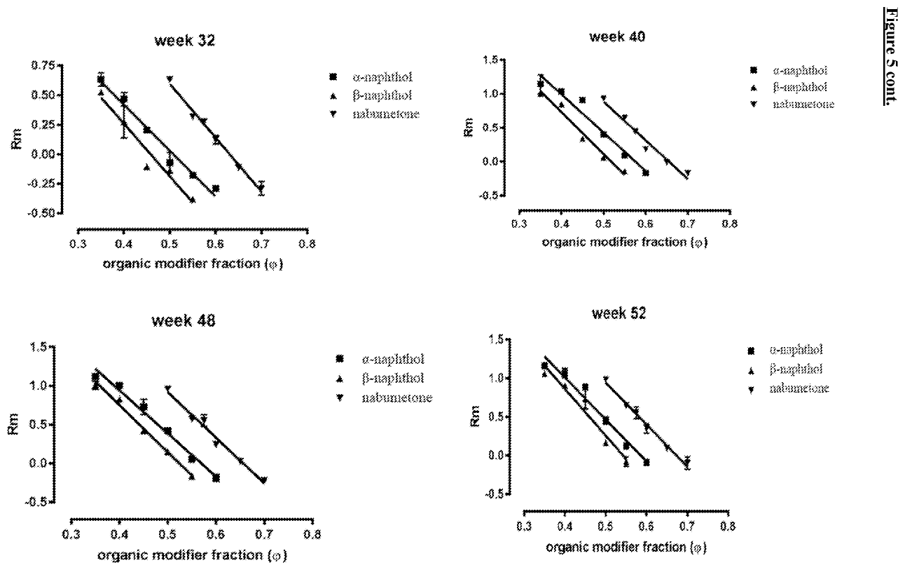

FIG. 7 is a schematic low diagram for classification of small molecule drugs from highly hydrophilic to highly lipophilic based on ICHI;

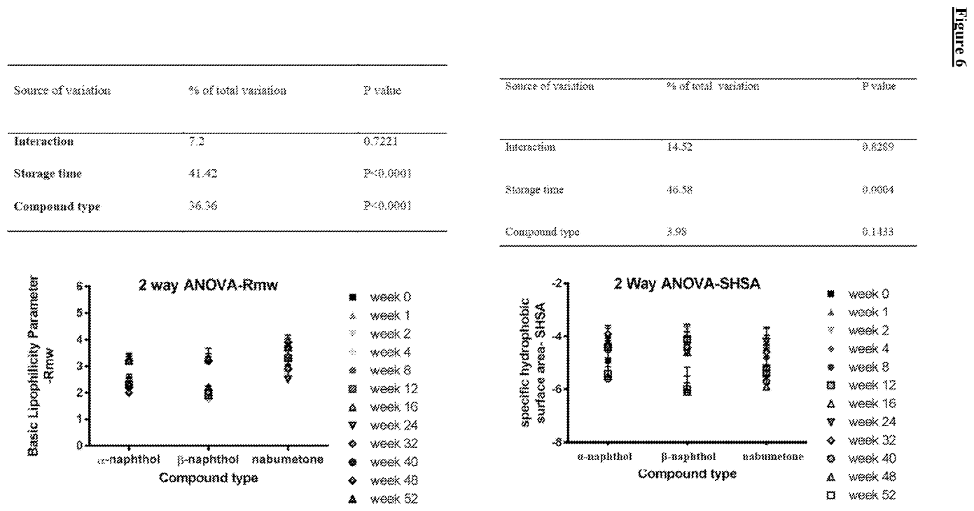

FIG. 8 is a schematic diagram showing the architecture of the small intestine;

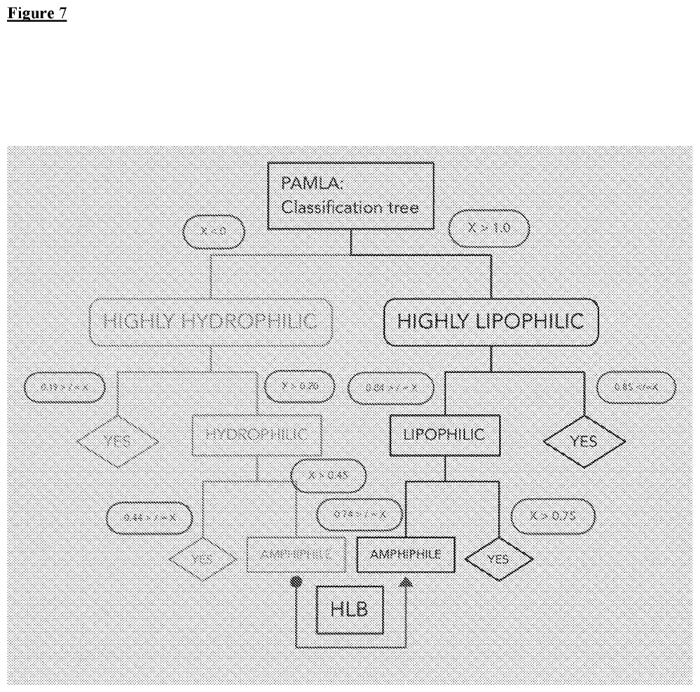

FIG. 9 is a schematic diagram showing the absorption pathway of the unstirred water layer of the small intestine;

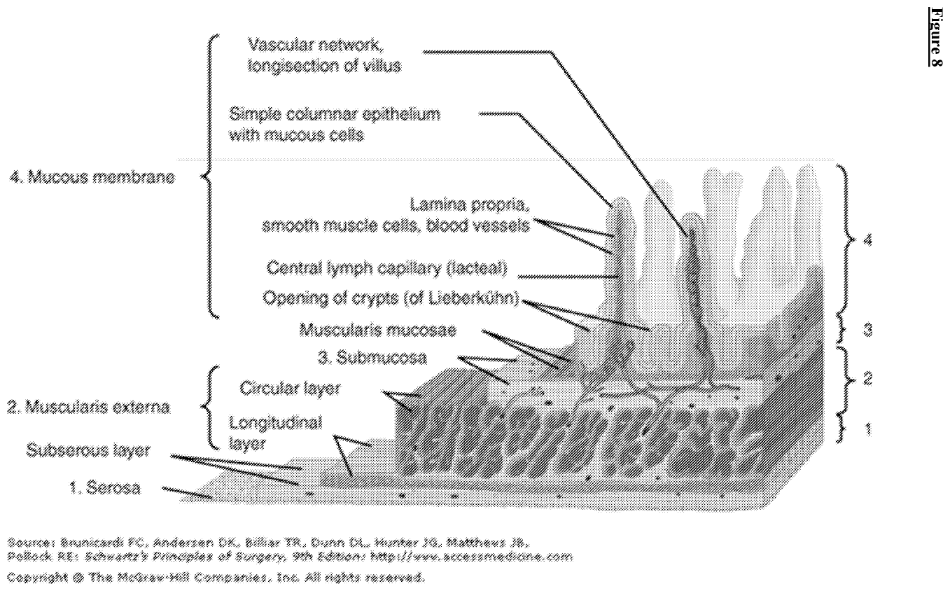

FIG. 10 is a schematic diagram showing the physics of hydration and flow regimen between LOD and ODS;

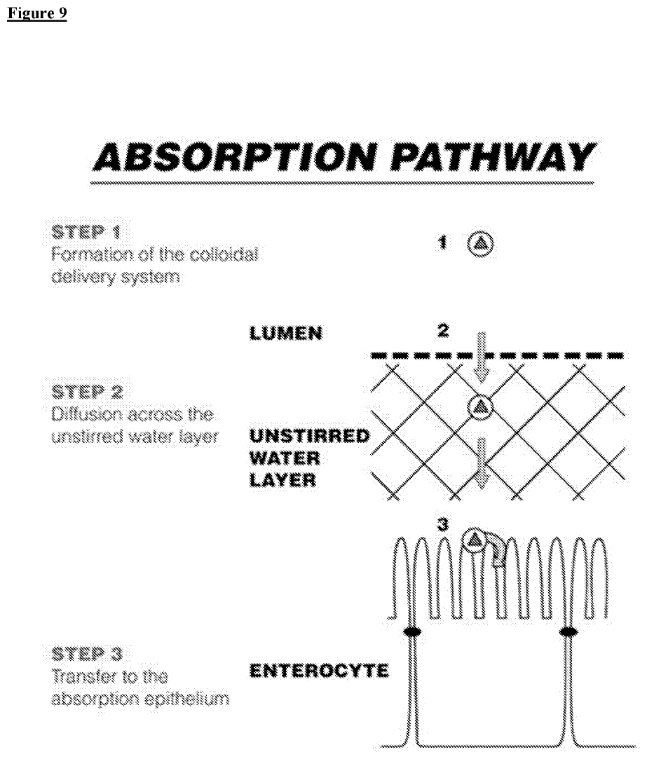

FIG. 11 are plot graphs of Rm versus MeOH fraction for caffeine and halofantrine for PAMLA and ODS;

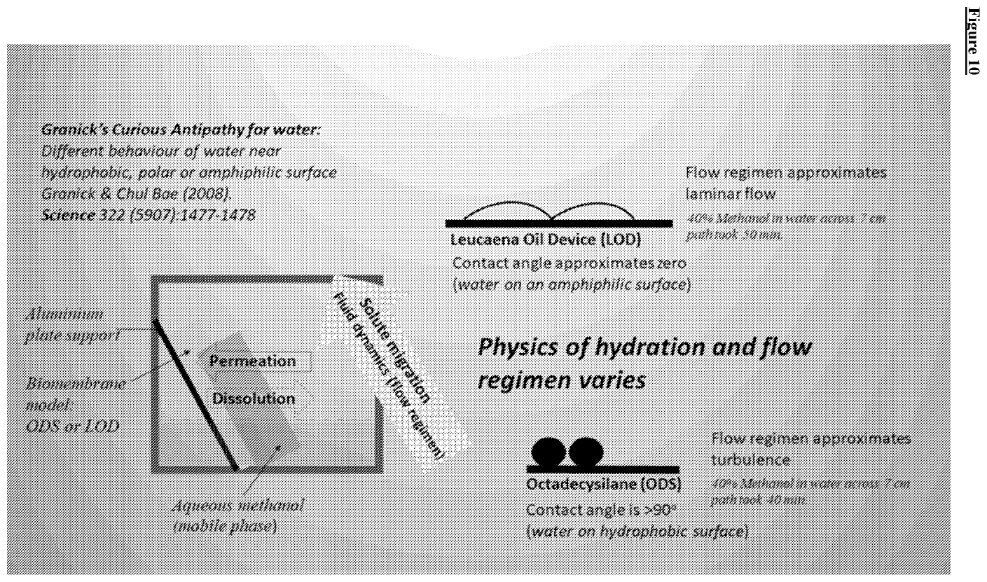

FIG. 12 are plot graphs of Rm_{w} for caffeine and halofantrine on ODS and PAMLA;

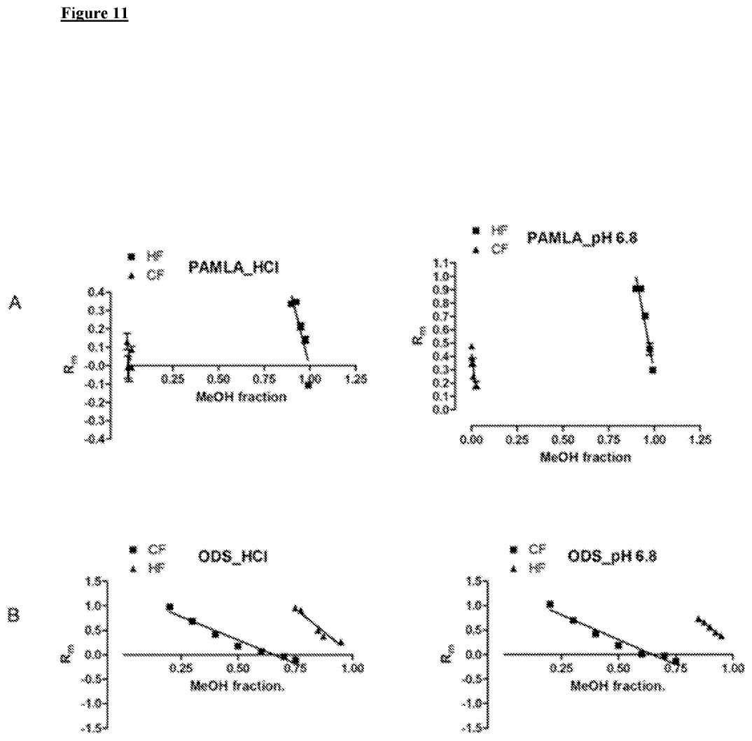

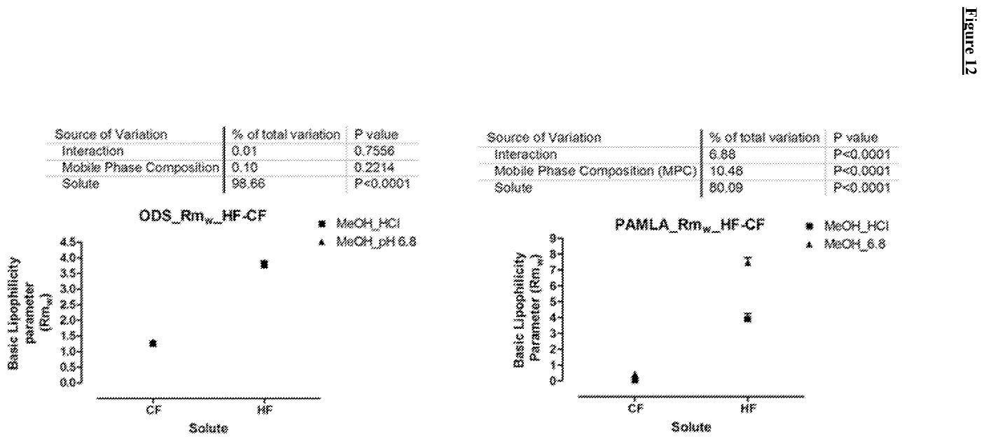

FIG. 13 are plot graphs of specific hydrophobic surface area for caffeine and halofantrine on ODS and PAMLA;

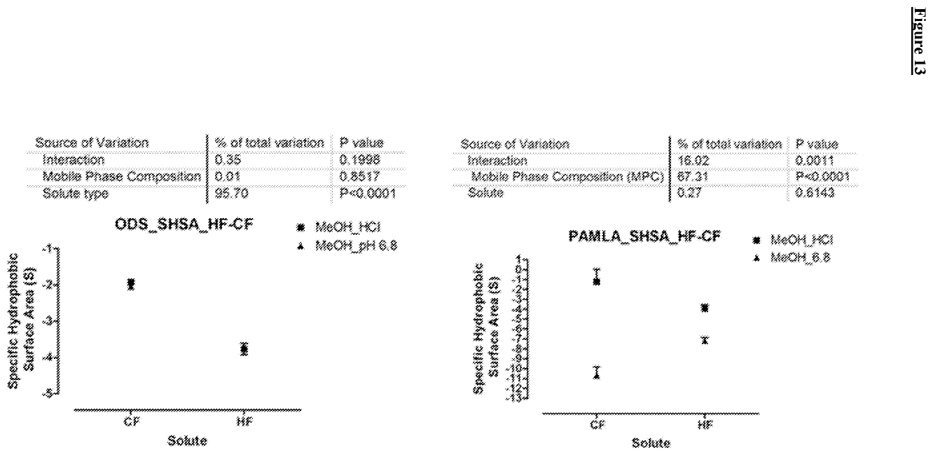

FIG. 14 are bar graphs of ICHI for caffeine and halofantrine on ODS and PAMLA;

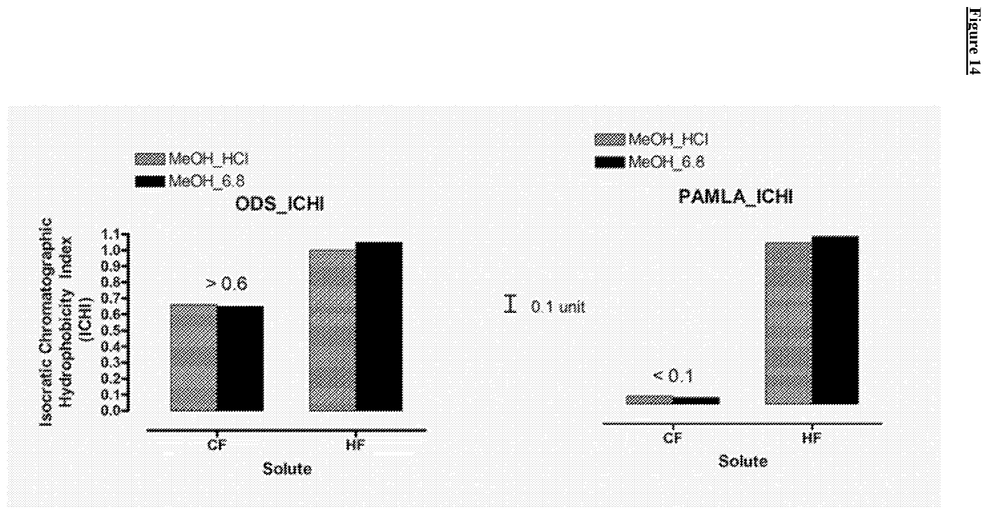

FIG. 15 are bar graphs of Qₛᵢ for caffeine and halofantrine on ODS and PAMLA;

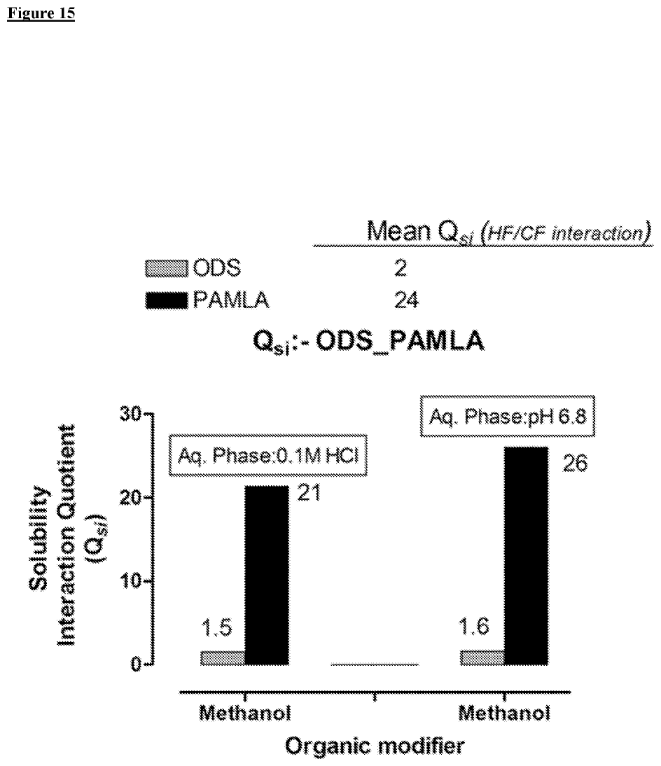

FIG. 16 shows chemical structures of caffeine and halofantrine;

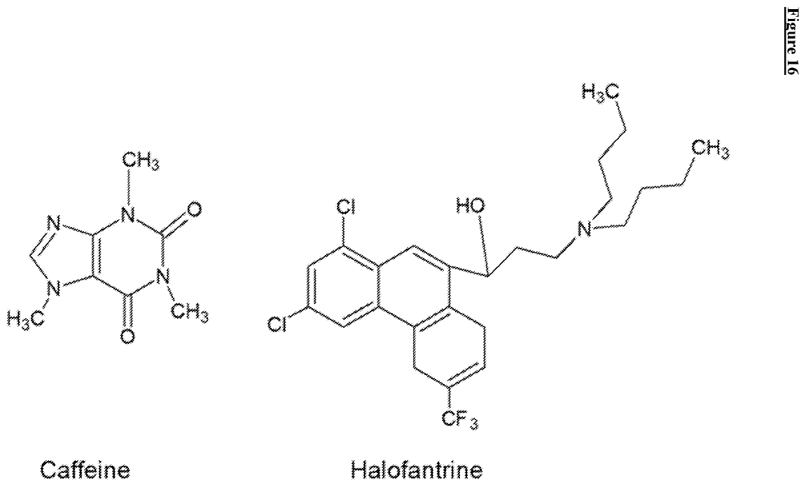

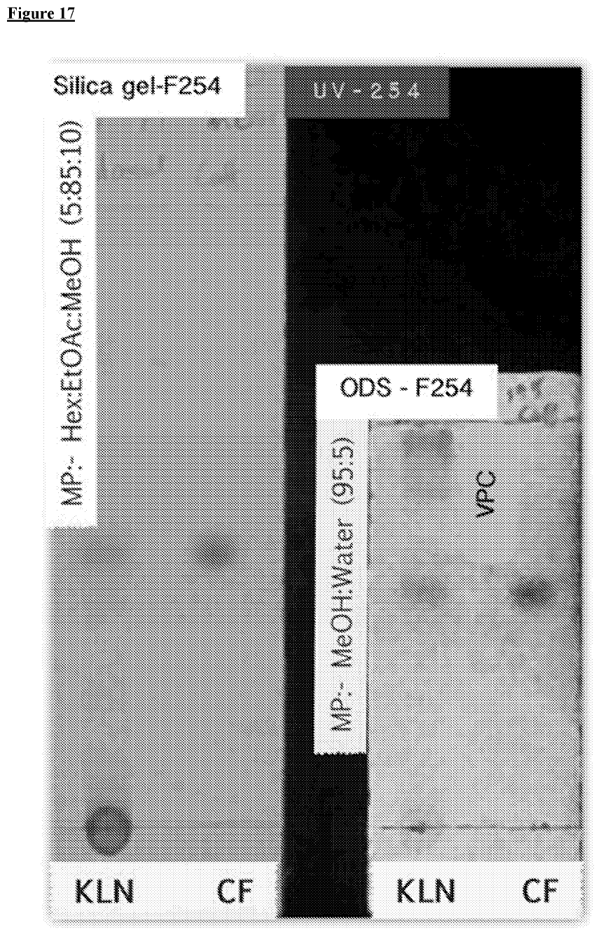

FIG. 17 shows a chromatogram of kolanut extract (KLN) and caffeine (CF) on silica gel GF₂₅₄ and ODS F₂₅₄;

FIG. 18 shows plot graphs of Rm versus methanol fraction for ketoconazole and halofantrine on ODS and LOD;

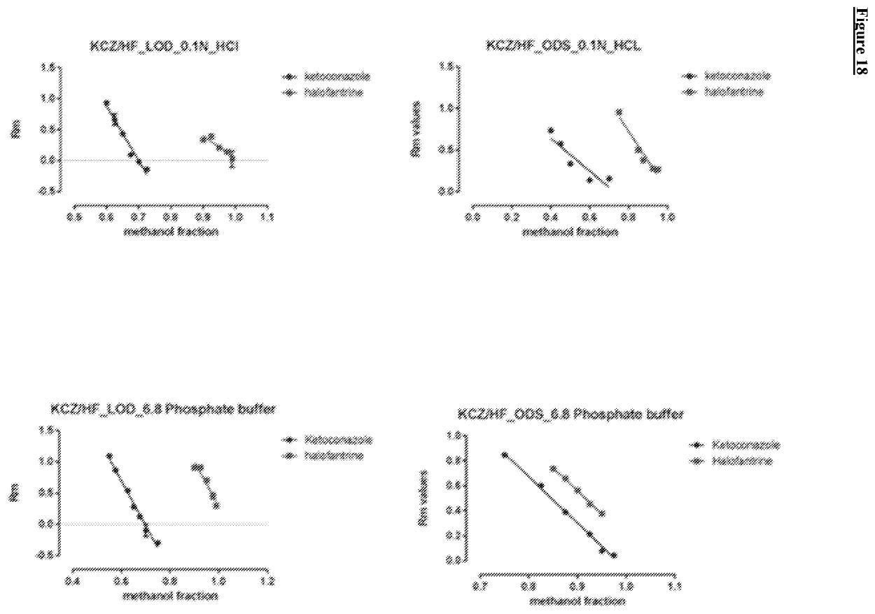

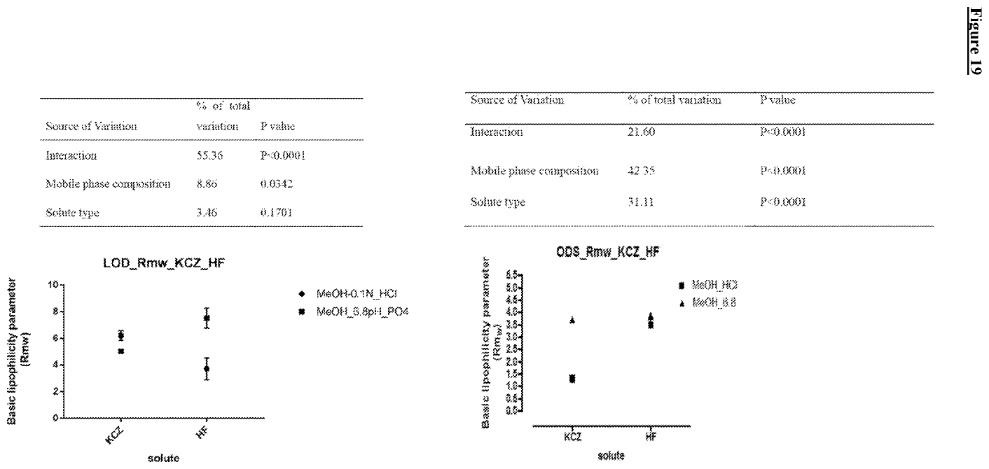

FIG. 19 shows box graphs of Rm_{w} for ketoconazole and halofantrine on ODS and LOD;

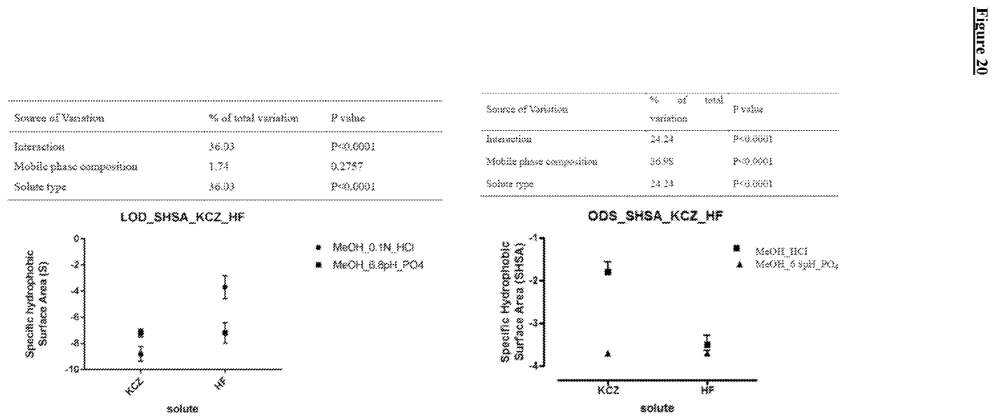

FIG. 20 shows box graphs of specific hydrophobic surface area for ketoconazole and halofantrine on LOD and ODS;

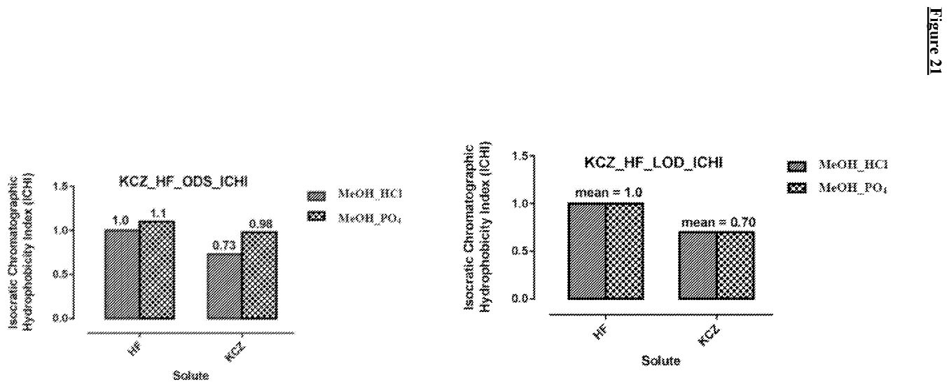

FIG. 21 shows box graphs of ICHI for ketoconazole and halofantrine on LOD and ODS;

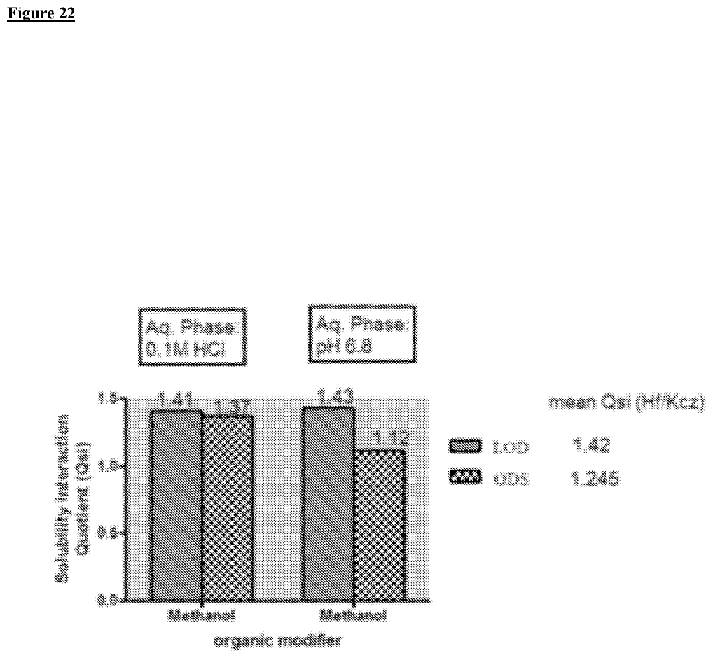

FIG. 22 shows bar graphs of Qₛᵢ for ketoconazole and halofantrine on LOD and ODS;

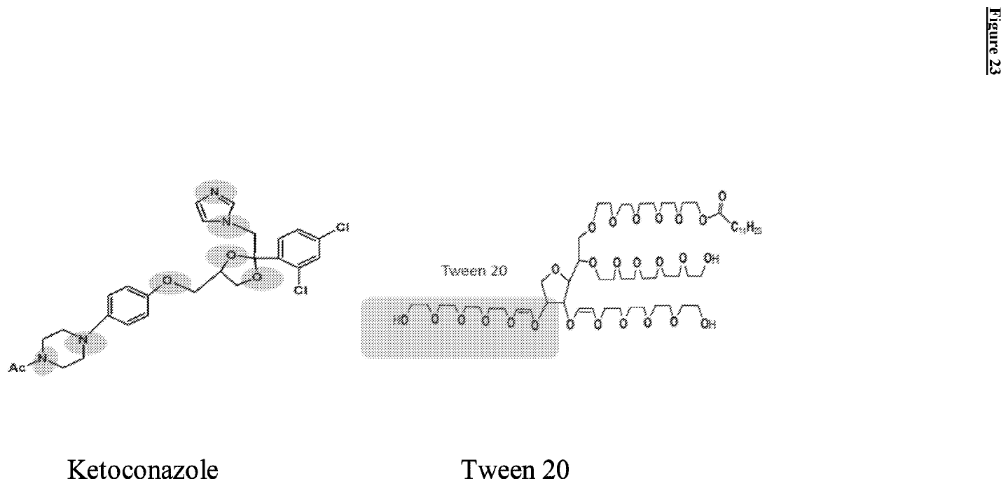

FIG. 23 shows chemical structures of ketoconazole and Tween 20; and

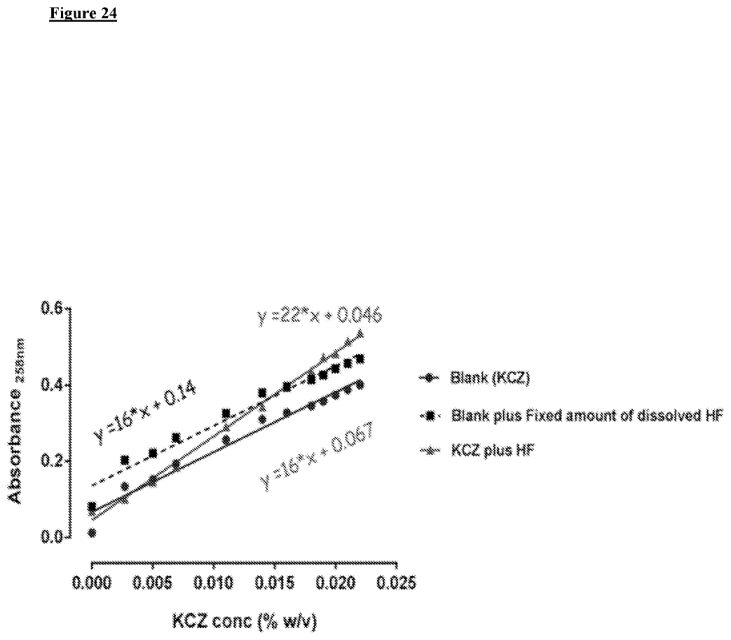

FIG. 24 shows a plot graph of absorbance versus ketoconazole (KCZ) concentration with and without halofantrine (IF).

### EXAMPLE 1

### Reliability of the Lipid Film Creation Process

The hypothesis was that lipid film thickness from run-to-run and batch-to-batch could vary significantly, owing to progressive evaporation of the volatile solvent (n-hexane) in which the coating solution is prepared and continuous utilization of the oil. Such variation may influence surface hydrophobicity (film thickness), solute retention behaviour and hence lipophilicity profiling of drug molecules.

The experiment aimed to discover how many runs can be done with a standard film creation set-up before sacrificing the integrity of the film surface.

### Standard Film Creation Set-Up

10 mL of 3.75% v/v solution of refined Leucaena oil was prepared in n-hexane. Silica gel GF₂₅₄ plates (5×10 cm) are successively developed by ascending development in the oil solution. Ten consecutive runs were carried out in each of the six tanks set up to prepare the Leucaena oil device (LOD).

The devices were labelled according to the number of runs used for their production, from the first run (R1) to the last run (R10).

As described above, the LOD was designed for lipophilicity measurements and was created and reported to provide a more accurate simulation of the bio-membrane. The small intestine has a layer of water, the Unstirred Water Layer (UWL), held in place by hydrogen bond network between the gut wall and luminal contents. A proper simulation of the UWL in a lipophilicity measuring device will represent a biomimetic attribute, while absence of a boundary layer will represent systematic error in the measuring device. FIG. 8 illustrates the relevant anatomy whereas FIG. 9 illustrates the absorption pathway of the UWL.

The following extract from Sugano (2012) compares the fluid dynamics between the LOD and ODS platforms:

Theoretic arguments and experimental data on flow regimen of 40% methanol (model mobile phase) across the two biomembrane models provide insight into the relative validity of the two measurement processes.

Fluid dynamics of fluid across a surface stipulates that a boundary layer exists when the flow regimen is laminar flow (simulation of Unstirred Water Layer, UWL), while the boundary layer is inapplicable when the flow is turbulent. The velocity of a mobile phase of the same composition and viscosity across the same path length (7 cm) is much higher on ODS (10 minutes difference). The higher velocity could lead to a higher Reynold's number that signifies transition from laminar to turbulent flow.

Considering this fact, physiological UWL is properly accounted for by LOD platform and the lipophilicity measurement on LOD is therefore more valid than measurement on ODS platform, where physiological UWL is not accounted for, constituting a systematic error, leading to a general overestimation of lipophilicity.

FIG. 10 summarises the physics of hydration and flow regimen for LOD and ODS. As well as its importance in accurately measuring the lipophilicity of individual molecules, it has been discovered that the LOD can also be useful in predicting drug-drug interactions and their effect on the bioavailability of drugs when co-administered with another drug or herbal supplement.

### Sample Spotting and Development of Chromatoplate

Four model compounds were used for the study; naproxen, β-naphthol, α-naphthol and nabumetone.

Solutions of the model compounds were prepared in methanol (0.025%) and applied to the plates (R1, R2, R3, R4, R5, R6, R7, R8, R9, R10, respectively) with 2 μL capillary tubes. The plates were air-dried at room temperature and the plates were developed in saturated development tanks containing 10 mL of the mobile phase. The plates were developed over a path of 7 cm and allowed to air-dry before visualization under UV light at 254 nm. The retardation factor (Rf) was then measured. The development was performed in duplicate.

### Reproducibility

The whole procedure was repeated after 2 weeks, using a different set of glassware in order to evaluate the impact of calibration and volume errors on batch-to-batch variation in film thickness of the lipid device. The four determinations for each data point were pooled together to give a mean Rm value.

### Mobile Phase

The mobile phase consisted of various concentrations of methanol in water. For naproxen (0.3125, 0.625, 1.25 and 2.5% v/v); α-naphthol (35, 45, 50, 55, 60, 65% v/v); β-naphthol (35, 40, 45, 50, 55, 60% v/v) and nabumetone (50, 55, 57.5, 60, 65, 70% v/v).

### Equations

Rm was computed by the equation:

$$Rm = Log ( 1 / Rf - 1 )$$

The linear regression of Rm versus organic modifier fraction is governed by the Soczewinski Wachmeister equation:

$$Rm = Rmw + {S}^{φ}$$

### Results & Discussion

As shown in FIG. 2, Naproxen exhibited a different retention behaviour on the device as shown by the cluster of data points collected for linear regression, relative to the other three compounds. This unique behaviour reflects the characteristics of naproxen as a highly hydrophilic compound. The lipophilicity metric is defined as the organic modifier required to obtain half-way maximum migration (Rf=0.5, Rm=0) for each of the drugs studied. This quantity is typically determined as the x-intercept of the linear regression of Rm against organic modifier fraction. The observation was that naproxen has a Rf>0.5 without any organic modifier in the mobile phase.

An attempt was made to obtain a linear plot by varying methanol fraction over a very narrow interval (0.3125 to 2.5%). It was shown that the slope of a linear plot could not be obtained reliably. The slope was not significantly non-zero. The Rm_{w} (y-intercept) could be determined experimentally (using water alone as the mobile phase) rather than graphically, which is the standard procedure for more lipophilic molecules.

The y-intercept, Rm_{w}, is the basic lipophilicity parameter, while the slope S, is the specific hydrophobic surface area (SHSA). This observation provided the teaching of determining the Rm_{w} experimentally for highly hydrophilic compounds (once Rf>0.5 is obtainable with distilled water alone as mobile phase) because SHSA could not be reliably determined in such cases.

This pattern is illustrated by FIGS. 3 and 4. As shown in FIG. 3, the slope of a linear plot (SHSA) could not be reliably determined for naproxen, because the basic lipophilicity parameter Rm_{w} could be determined using water alone as mobile phase (Rf>0.5 is obtainable, Rf of 0.75 in water gives Rm_{w} value of −0.5). As shown in FIG. 4, consistent ICHI values were obtained for the 3 molecules α-naphthol, β-naphthol and nabumetone, while naproxen shows inconsistency in the values obtained across the brands of LOD R1 to R10. The ICHI values are summarised below:
For α-naphthol: ICHI varies between 0.52-0.54
For β-naphthol: ICHI varies between 0.46-0.48
For nabumetone: ICHI varies between 0.61-0.63
   For naproxen: ICHI varies widely between −0.065-−5.8 (including some positive values)

Taken together, the exceptionally large value given by R10 for NPX, and the general difficulty associated with the preparation of R10 (coating solution was considerably smaller, owing to progressive volume reduction and little evaporation), it is preferable to do a maximum of 9 runs with a standard set-up of 10 mL LO in n-hexane (3.75%) coating solution.

FIG. 7 and Table 1 disclose an algorithm for the classification of small molecule drugs based on isocractic chromatographic hydrophobicity index (ICHI).

TABLE 1

| Classification | ICHI |
|---|---|
| Highly hydrophilic | ICHI ≤ 0.19 |
| Hydrophilic | 0.19 < ICHI ≤ 0.44 |
| Amphiphilic | 0.44 < ICHI ≤ 0.74 |
| Lipophilic | 0.74 < ICHI < 0.85 |
| Highly lipophilic | 0.85 ≤ ICHI |

Table 2 discloses the predicted effect of the co-administration of a secondary active compound on the bioavailability of a primary active compound based on the solubility interaction quotient (Qₛᵢ).

TABLE 2

| | Qₛᵢ | | |
|---|---|---|---|
| Predicted effect | Method 1 | Method 2 | Method 3 |
| Reduced | Qₛᵢ > about 2 | Qₛᵢ > about 2.3 | Qₛᵢ ≥ about 2.27 |
| bioavailability | | | |
| Enhanced | Qₛᵢ = about 1 − | Qₛᵢ = about 1.4 − | About 1.35 ≤ |
| bioavailability | about 2 | about 2.2 | Qₛᵢ < about 2.27 |
| No substantial | Qₛᵢ = about 1 | Qₛᵢ < about 1.3 | Qₛᵢ < about 1.35 |
| effect on | | | |
| bioavailability | | | |

### EXAMPLE 2

### Reliability of Lipid Film Integrity on Storage

It was hypothesized that the lipid film surface chemistry could be modified on storage by lipid peroxidation. Oxidation of the lipid system would alter its hydrophobicity and hence the partition dynamics, leading to deviation of lipophilicity measurement from the behaviour of freshly-prepared device.

### Reliability of LOD on Storage

The device was prepared as previously described in Example 1, and stored at different time intervals of 1, 2, 4, 8, 12, 16, 24, 32, 40, 48, and 52 weeks, respectively.

The freshly prepared device (week 0) and other stored devices were used in turn to profile the lipophilicity of α-naphthol, β-naphthol and nabumetone as previously described. The model parameters Rm_{w} and SHSA were compared by 2-way ANOVA to detect any deviation in performance between the stored device relative to the freshly prepared one. Bonferroni post ANOVA test was used to determine the point at which deviation in performance began.

### Results & Discussion

FIGS. 5 and 6 show that retention behaviour on stored devices fits into two distinct clusters; 0-32 weeks and 40-52 weeks. The two model parameters, Rm_{w} and SHSA, were higher on devices stored for 40-52 weeks, suggesting that extensive lipid peroxidation renders the lipid surface more hydrophobic.

2-way analysis of variance evaluated the impact of storage time and compound type on the determined model parameters, namely, Rm_{w} (basic lipophilicity parameter) and S (specific hydrophobic surface area, SHSA). It was shown that storage time was the most important source of variation, responsible for 41% of variation in Rm_{w} and 47% of variation in SHSA.

It was shown by post ANOVA Bonferonni test and evident that there is better agreement between the performance of freshly prepared device and the devices stored up to 32 weeks. By 40 weeks and beyond, there is evidence of device failure. In the graphical plot below, the model parameters are seen in 2 clear clusters (cluster 1 is 0-32 weeks and cluster 2 is 40-52 weeks). In general, Rm_{w} and SHSA values were higher at week 40 and beyond, leading to higher values for the lipophilicity metric. The overall impact of storage beyond 32 weeks was that the surface chemistry of the lipid device becomes more hydrophobic.

The physics of hydration and fluid dynamics across the surface of 40 weeks old device is different than for freshly prepared device, hence the difference in performance. Lipid peroxidation is reported to form chemiluminescent intermediate products such as singlet oxygen and excited triplet carbonyl species. The physics of hydration exhibited by these species led to greater fluctuations in interactions of water with the surface. The existence of a boundary layer in the course of chromatographic development and the laminar flow of aqueous mobile phase across the surface are interfered with. The retention behaviour on 40 weeks old device therefore approximates the retention behaviour observed on the hydrocarbon bonded phase, octaldecylsilane (ODS). This observation is an unexpected pattern.
Failure mode: Extensive lipid peroxidation on storage.
Failure effect: The hydrophobicity of the lipid surface was altered by lipid peroxidation, with the resultant effect being a higher hydrophobicity.
Criticality analysis: An automated failure detection system is built in the device. Self-assembled UV active phytochemicals in the lower zone of the device exhibit chemiluminescence after oxidation. A bright blue glow in the lower zone under UV-366 nm is more intense than the weak chemiluminescence that characterises the oxidation of the lipid system in general, which is evident over the entire surface of the device on storage beyond 32 weeks.

Whatever degree of environmental stress is imposed on the device on storage, the self-assembled, built-in oxygen sensors (BIOS) constitute an automated failure detection system to detect device degradation, which signifies that the device is inoperable (TESTABILITY).

It was concluded that the LOD could be re-used without failure after being stored for a maximum of 32 weeks at room temperature (ca. 33.8=1.8° C.). Beyond 32 weeks, loss of function occurred as a result of extensive lipid peroxidation, which led to an unexpected discovery that the surface was actually rendered more hydrophobic.

### EXAMPLE 3

De-Wetting of Halofantrine from Drug-Drug Interaction

Kolade et al. (2008) disclosed that concomitant intake of kolanut with halofantrine significantly decreased Cₘₐₓ and AUC of both halofantrine and the metabolite, desbutylhalofantrine, while no significant effect was observed for tₘₐₓ and t_{1/2} of the compounds.

Kolanut contains caffeine and other very polar constituents (VPC), while halofantrine is a highly lipophilic drug.

The inventors hypothesised that co-administration of caffeine and VPC in kolanuts (hydrophilic compounds) with halofantrine (highly lipophilic and poorly water soluble) will competitively inhibit the hydration of halofantrine and induce de-wetting. It was thought that de-wetting could considerably reduce dissolution rate, and hence, the absorption and bioavailability of halofantrine, which is normally limited by dissolution.

This experiment aimed to find evidence that caffeine is notably hydrophilic relative to lipophilic halofantrine, such as to create this impact on halofantrine dissolution rat.

### Reversed Phase Thin Layer Chromatography

### ODS Platform

The mobile phase used consisted of various concentrations of methanol in 0.1N HCl and phosphate buffer pH 6.8. For caffeine (20, 30, 40, 50, 60 and 70% v/v); and halofantrine (75, 85, 87.5, 90, 92.5 and 95% v/v).

### LOD Platform

The mobile phase used consisted of various concentrations of methanol in 0.1N HCl and Phosphate buffer pH 6.8. For caffeine (0.1, 0.25, 0.5, 1.0, 2.5 and 5.0% v/v); and halofantrine (90, 92.5, 95, 97.5, 99% v/v).

### Data Analysis

Data analysis was done as previously described, Rf was transformed to Rm and linear regression analysis implemented to determine the lipophilicity parameter, ICHI.

### Results

FIG. 11 shows a linear regression demonstrating the retention behaviour of caffeine and halofantrine on the ODS and LOD (PAMLA) platforms. FIG. 12 shows that, with respect to Rm_{w}, only solute type (99%) is significant on ODS, while solute type (80%), mobile phase composition (10.5%) and interaction (7%) between the factors are all significant on LOD. FIG. 13 shows that, with respect to SHSA, only solute type (96%) is significant on ODS platform, whereas on LOD, solute type is not significant, rather mobile phase composition (67%) is the most significant factor and, to a lesser extent (16%), the interactions. FIG. 14 shows the lipophilicity of caffeine and halofantrine as measured by ODS and LOD platforms. On ODS, caffeine is judged amphiphilic, with ICHI>0.6, while LOD judged caffeine as highly hydrophilic, with ICHI<0.1. This great disparity signifies a fundamental difference in the partition dynamics on the two platforms.

### Solubility Interaction Quotient (Qₛᵢ)

A computational model and a composite metric for predicting most likely outcome of drug/drug interaction when two drugs are co-administered. The metric is a ratio of the lipophilicity metric, ICHI, of the more lipophilic drug to that of the less lipophilic drug.

$${Q}_{si} = \frac{{Φ}_{{O}_{HF}}}{{Φ}_{{O}_{CF}}} = \frac{{Rmw}_{HF} \cdot {S}_{CF}}{{S}_{HF} \cdot {Rmw}_{CF}}$$

FIG. 15 shows a great disparity in the solubility interaction quotient prediction arising from the classifications of caffeine on ODS and LOD. On ODS, halofantrine is judged to be only 2-fold more lipophilic than caffeine, whereas on LOD, halofantrine is judged 24-fold more lipophilic.

### Structural Theory and Measured Lipophilicity

FIG. 16 shows the chemical structures of caffeine and halofantrine. Caffeine has polar surface area (PSA) of 58.44, while halofantrine has a PSA of 23.47. The lipophilicity measured on LOD appears more consistent with structural theory than the measurement produced by ODS. It is evident that ODS has a systematic error. FIG. 17 is a chromatogram showing kolanut extract (KLN) and caffeine (CF) on silica Gel GF₂₅₄ and ODS F₂₅₄. This shows that kolanut extract contains very polar constituents (VPC) that are even more polar than caffeine, alongside caffeine.

### Discussion

It is evident that kolanut extracts contain VPC that are far more hydrophilic than caffeine. Using caffeine as probe of hydrophilic constituents of kolanut, we can argue that the VPC will have Qₛᵢ>24.

This finding is consistent with a hypothesis that co-administration of kolanut extract with halofantrine would lead to de-wetting of halofantrine, thus reducing its dissolution rate and hence bioavailability.

The lowering of halofantrine bioavailability (AUC) and Cₘₐₓ observed when kolanut extract was co-administered with it can be reliably predicted by the measurement made on LOD but not by measurement made on ODS. LOD is thus a biomimetic platform, ensuring biorelevance of lipophilicity measurement, while ODS is not biomimetic and hence, not a valid model of the biomembrane.

The physics of hydration at the amphiphilic surface and a flow regimen that approximates laminar flow, rather than turbulent flow, support the existence of a boundary layer, which is a simulation of the UWL, on the LOD platform. The absence of a boundary layer on ODS platform means a systematic error exists on this platform and ODS invariably overestimates the lipophilicity of small molecule drugs.

It was concluded that the LOD platform can be applied to experimental therapeutics through prediction of most likely solubility interaction outcome when 2 drugs or drug and dietary supplements are co-administered.

### EXAMPLE 4

Micellar Solubilisation of Halofantrine from Drug-Drug Interaction

Milton et al. (1989) disclosed that the oral bioavailability of halofantrine (Hf) was increased 3-fold in humans and 12-fold in dogs, when administered postprandially; however, the proportional formation of the active desbutyl metabolite (desbutylhalofantrine, Hfm) decreased 2.4-fold in humans and 6.8-fold in dogs.

In Khoo et al. (1998), a study was undertaken to confirm the putative involvement of CYP3A4 in the N-dealkylation of Hf to Hfm by administering Hf with and without ketoconazole, a specific CYP3A4 inhibitor, and measuring the resulting plasma concentration profiles of Hf and Hfm. The plasma Hfm/Hf AUC (0-72 h) ratio after fasted oral administration of Hf without ketoconazole was 0.56, whereas the ratio after fasted oral administration with ketoconazole was less than 0.05. It is likely that both hepatic and prehepatic (enterocyte-based) CYP3A4 contributed to metabolism of Hf to Hfm after oral administration. Interestingly, the low plasma Hfm/Hf AUC ratios observed after fasted administration of Hf with ketoconazole were similar to the low values previously observed when Hf was administered postprandially (despite increased Hf absorption). The mechanism(s) by which postprandial administration of Hf led to a decrease in its metabolism is/are unknown.

The inventors hypothesised that structural theory predicts that the alternating polar area in hydrophobic structural motif found in ketoconazole is like the structure of non-ionic surfactants (Tweens) and could make ketoconazole an amphiphile with surfactant-like property.

This study was undertaken to determine whether it is possible to predict an absorption level of interaction between ketoconazole and halofantrine based on their physicochemical properties and HLB-sensitive lipophilicity classification and to find evidence that ketoconazole could enhance the dissolution and hence, the absorption of halofantrine (along with its action as a specific inhibitor of CYP3A4 enzymes), through thermodynamic solubility experiments.

### Mobile Phase Preparation for Reversed Phase Chromatography

The mobile phase used in this work consisted of various concentrations of methanol in phosphate buffer (pH 6.8) and 0.1N HCl, depending on the stationary phase used, i.e. 60, 62.5, 65, 67.5, 70, 72.5% v/v (ketoconazole_LOD-0.1N HCl); 55, 57.5, 62.5, 65, 67.5, 70, 75% v/v for Ketoconazole on LOD-phosphate buffer platform; 40, 45, 50, 60, 70% v/v for ketoconazole on Octadecylsilane (ODS)-0.1N HCl; 75, 82.5, 87.5, 92.5, 95, 97.5% v/v for ketoconazole on Octadecylsilane (ODS)-phosphate buffer platform; while 90, 92.5, 95, 97.5, 99% v/v for Halofantrine on LOD-0.1N HCl and LOD-Phosphate buffer platform; 75, 85, 87.5, 92.5, 95% v/v for Halofantrine on Octadecylsilane (ODS)-0.1N HCl; and 85, 87.5, 90, 92.5, 95% v/v for Halofantrine on Octadecylsilane (ODS)-Phosphate buffer platform.

### Data Analysis

Data analysis was performed as described earlier under data analysis in previous sections. Rf is transformed to Rm and linear regression of Rm versus methanol fraction produces linear regression parameters that provide the lipophilicity metric.

### Results

FIG. 18 shows a linear regression demonstrating the retention behaviours of ketoconazole and halofantrine on the ODS and LOD (PAMLA) platforms. FIG. 19 shows that, with respect to Rmw, all the factors are significant on ODS, with mobile phase composition contributing the highest (42%) to the variation, whereas on LOD, interactions between the factors is the most significant (55%) source of variation. FIG. 20 shows that, with respect to SHSA, solute type and interactions between the factors are significant sources of variation for the two platforms, but mobile phase composition is only significant on the ODS platform.

FIG. 21 shows the lipophilicity of halofantrine and ketoconazole on the ODS and LOD platforms showing greater consistency of measures on LOD with variation in mobile phase composition. Using mean values, LOD classifies ketoconazole as amphiphilic (ICHI=0.70), while ODS classifies ketoconazole as lipophilic (ICHI=0.86). This finding signifies significant difference in partition dynamics on the two platforms. FIG. 22 shows that the Qₛᵢ from LOD exclusively predicts that interaction of ketoconazole and halofantrine will lead to enhancement of halofantrine dissolution through micellar solubilization (surfactant-like) property. The reference range for this prediction is 1.35-2.22 and a value of 1.42 was obtained on LOD.

### Structural Theory and Measured Lipophilicity

FIG. 23 shows the chemical structures of ketoconazole and Tween 20. The structure of ketoconazole is like the structure of Tween 20 in alternating pattern of polar area within hydrophobic moieties. The measurement of lipophilicity on LOD is consistent with the hypothesis that ketoconazole is amphiphilic in nature.

FIG. 24 shows the concentration-dependent paradoxical effect of ketoconazole on halofantrine solubility (0.015% being the critical point, i.e. <0.015%, KCZ decreases HF solubility, while >0.015% KCZ increases HF solubility). Ketoconazole appears to exhibit a ‘critical micellar concentration’ characteristic of surfactants around 0.015%, where the plot of experimental KCZ plus halofantrine (over 48 hours incubation) versus absorbance intersects the hypothetical line obtained from adding a fixed absorbance value (corresponding to a fixed amount of dissolved halofantrine in the absence of KCZ) to each blank data point.

### Discussion

Lipophilicity measurement on LOD platform classified ketoconazole as an amphiphilic molecule, and a surfactant-like property was predicted based on that classification. The surfactant-like property was demonstrated by thermodynamic solubility experiments, which showed that beyond a certain critical concentration, ketoconazole enhanced the solubility of halofantrine. This enhanced solubility would lead to enhanced dissolution rate and increased bioavailability for halofantrine.

It was therefore demonstrated that absorption-level interaction co-exists with metabolic level of interaction previously documented when ketoconazole is co-administered with halofantrine.

It was concluded that lipophilicity measurement and classification of small molecules by LOD has led to an unexpected discovery of absorption-level drug/drug interaction. This model can be extended to experimental therapeutics for discovery of unexpected drug/drug interaction.

### EXAMPLE 5

### De-Wetting of Ciprofloxacin Hydrochloride

Esimone et al. (2002) discloses an in vivo evaluation of the interaction between aqueous seed extract of Garcinia kola Heckel and the antibiotic ciprofloxacin hydrochloride. The evaluation revealed that co-administration of aqueous extract of Garcinia kola seed (which is rich in bioflavonoids) reduces the bioavailability of ciprofloxacin hydrochloride 1-hour post-administration. A biphasic interaction pattern was identified in which the bioavailability of ciprofloxacin hydrochloride later increased following the initial reduction.

The inventors hypothesised that the initial reduction in the bioavailability of ciprofloxacin hydrochloride was caused by de-wetting of ciprofloxacin hydrochloride by the bioflavonoids in the aqueous extract of Garcinia kola seed leading to a reduction of the dissolution rate and hence bioavailability of ciprofloxacin hydrochloride.

To test this hypothesis, the inventors calculated the Qₛᵢ of the combination of ciprofloxacin hydrochloride and model Garcinia bioflavonoid using LOD. The combination has a Qₛᵢ of about 10. Using the method of the invention, this Qₛᵢ value would lead to a prediction that the co-administration of aqueous extract of Garcinia kola seed would reduce the bioavailability of ciprofloxacin hydrochloride. This prediction is consistent with the results of the in vivo evaluation disclosed in Esimone et al. (2002) which further validates the method of the invention.

It is possible that other components of the aqueous extract of Garcinia kola seed, apart from bioflavonoids, can exhibit other effect profiles that could explain the later enhancement of the bioavailability of ciprofloxacin hydrochloride. To this end, additional studies are ongoing to fully elucidate what more could be predicted from chromatographic lipophilicity experiments.

### REFERENCES

Cross et al. 2003. Determination of the effect of Lipophilicity on the in-vitro permeability and tissue reservoir characteristics of topically applied solutes in human skin layers. Journal of Investigative Dermatology. 120.5:759-764.
Esimone et al. 2002. In vivo evaluation of interaction between aqueous seed extract of Garcinia kola Heckel and ciprofloxacin hydrochloride. Am J Ther. 9 (4): 275-80.
Idowu et al. 2009. Engineering and validation of a novel lipid thing film for biomembrane modeling in lipophilicity determination of drugs and xenobiotics. Journal of Biological Engineering. 3:14 doi: 10.1186/1754-1611-3-14.
Kaliszan et al. 2003. New approaches to chromatographic determination of lipophilicity of xenobiotics. Analytical and Bioanalytical Chemistry. 377:803-811.
Kerns et al. 2003. Pharmaceutical profiling in drug discovery. Drug Discovery Today. 8:316-323.
Khoo et al. 1998. Metabolism of halofantrine to its equipotent metabolite desbutylhalofantrine, is decreased when orally administered with ketoconazole. Journal of Pharmaceutical Sciences. 87.12:1538-1541.
Kolade et al. 2008. Effect of kolanut on the pharmacokinetics of the antimalarial drug halofantrine. Eur. J Clin Pharmacol. 64 (1): 77-81.
Lipinski et al. 2000. Drug-like properties and the causes of poor solubility and poor permeability. Journal of Pharmacology and Toxicology Methods. 44:235-249.
Milton et al. 1989. Br. J. Clin. Pharmacol. 28:71-77.
Penzotti et al. 2004. Building predictive ADMET models for early decisions in drug discovery. Current Opinion in Drug Discovery and Development. 7:49-60.
Rick et al. 2009. Drugs: From Discovery to Approval. New Jersey: Wiley-Blackwell Publishers pg 3.
Serda et al. 2012. Investigation of the biological properties of (hetero) Aromatic Thiosemicarbazones. Molecules. 17:13483-13502.
Sugano. 2012. Fluid dynamics. In: Biopharmaceutics modelling and simulations, Theory, Practice, Methods and Applications. pp 468-476, John Wiley & Sons, New Jersey, USA.

1. A method for predicting the effect of the co-administration of a secondary active compound on the bioavailability of a primary active compound in an individual, the method comprising:
(d) calculating the solubility interaction quotient (Qₛᵢ) for the combination of primary and secondary active compounds according to the formula:
$${Q}_{si} = \frac{{ICHI}_{1}}{{ICHI}_{2}}$$
wherein ICHI₁ is the isocratic chromatographic hydrophobicity index (ICHI) of the primary active compound and ICHI₂ is the ICHI of the secondary active compound; and
(e) characterising the predicted effect of the co-administration of the secondary active compound on the bioavailability of the primary active compound based on the calculated Qₛᵢ value as either:
(i) reducing the bioavailability;
(ii) enhancing the bioavailability; or
(iii) having substantially no effect on the bioavailability.

2. The method of claim 1, wherein the ICHI values of the primary and secondary active compounds are measured using a Leucaena oil device.

3. The method of claim 1, wherein the predicted effect of the co-administration of the secondary active compound on the bioavailability of the primary active compound is characterised as:
(iv) reducing the bioavailability if Qₛᵢ is greater than about 2;
(v) enhancing the bioavailability if Qₛᵢ in the range of about 1 to about 2; or
(iii) having substantially no effect on the bioavailability if Qₛᵢ is equal to about 1.

4. The method of claim 1, wherein the predicted effect of the co-administration of the secondary active compound on the bioavailability of the primary active compound is characterised as:
(iv) reducing the bioavailability if Qₛᵢ is greater than about 2.3;
(v) enhancing the bioavailability if Qₛᵢ in the range of about 1.4 to about 2.2; or
(iii) having substantially no effect on the bioavailability if Qₛᵢ less than about 1.3.

5. The method of claim 1, wherein the predicted effect of the co-administration of the secondary active compound on the bioavailability of the primary active compound is characterised as:
(iv) reducing the bioavailability if Qₛᵢ≥about 2.27;
(v) enhancing the bioavailability if about 1.35≤Qₛᵢ<about 2.27; or
(vi) having substantially no effect on the bioavailability if Qₛᵢ<about 1.35.

6. The method of claim 1, wherein the primary and/or secondary active compounds comprise small molecules.

7. The method of claim 6, wherein a small molecule has a molecular weight of ≤900 Da.

8. The method of claim 1, wherein the primary active compound is lipophilic.

9. The method of claim 1, wherein the method is computer-implemented.

10-14. (canceled)

15. An apparatus for predicting the effect of the co-administration of a secondary active compound on the bioavailability of a primary active compound in an individual, the apparatus comprising:
(c) a Leucaena oil device;
(d) means for calculating the Qₛᵢ for the combination of primary and secondary active compounds according to the formula:
$${Q}_{si} = \frac{{ICHI}_{1}}{{ICHI}_{2}}$$
wherein ICHI₁ is the isocratic chromatographic hydrophobicity index (ICHI) of the primary active compound and ICHI₂ is the ICHI of the secondary active compound as measured by the Leucaena oil device; and
(f) means for characterising the predicted effect of the co-administration of the secondary active compound on the bioavailability of the primary active compound based on the calculated Qₛᵢ value as either:
(iv) reducing the bioavailability;
(v) enhancing the bioavailability; or
(vi) having substantially no effect on the bioavailability.

16. The apparatus of claim 15, wherein the predicted effect of the co-administration of the secondary active compound on the bioavailability of the primary active compound is characterised as:
(iii) reducing the bioavailability if Qₛᵢ is greater than about 2;
(iv) enhancing the bioavailability if Qₛᵢ in the range of about 1 to about 2; or
(iii) having substantially no effect on the bioavailability if Qₛᵢ is equal to about 1.

17. The apparatus of claim 15, wherein the predicted effect of the co-administration of the secondary active compound on the bioavailability of the primary active compound is characterised as:
(iii) reducing the bioavailability if Qₛᵢ is greater than about 2.3;
(iv) enhancing the bioavailability if Qₛᵢ in the range of about 1.4 to about 2.2; or
(iii) having substantially no effect on the bioavailability if Qₛᵢ less than about 1.3.

18. The apparatus of claim 15, wherein the predicted effect of the co-administration of the secondary active compound on the bioavailability of the primary active compound is characterised as:
(iv) reducing the bioavailability if Qₛᵢ≥about 2.27;
(v) enhancing the bioavailability if about 1.35≤Qₛᵢ<about 2.27; or
(vi) having substantially no effect on the bioavailability if Qₛᵢ<about 1.35.

19. The apparatus of claim 15, wherein the primary and/or secondary active compounds comprise small molecules.

20. The apparatus of claim 419, wherein a small molecule has a molecular weight of ≤900 Da.

21. The apparatus of claim 15,
wherein the primary active compound is lipophilic.

22. The apparatus of claim 15, wherein the apparatus further comprises a computer or central processing unit to calculate the Qₛᵢ value.
